(19) United States
(12) Patent Application Publication
(10) Pub. No.: US 2019/0122750 A1
(43) Pub. Date: Apr. 25, 2019

(54) AUTO-POPULATING PATIENT REPORTS

(71) Applicant: KONINKLIJKE PHILIPS N.V., EINDHOVEN (NL)

(72) Inventors: Erina Ghosh, Boston, MA (US); Eric Thomas Carlson, New York, NY (US); Oladijemi Feyisetan Farri, Yorktown Heights, NY (US)

(21) Appl. No.: 16/094,543

(22) PCT Filed: Apr. 13, 2017
(86) PCT No.: PCT/EP2017/058895

§ 371 Date: Oct. 18, 2018

Related U.S. Application Data
(60) Provisional application No. 62/324,949, filed on Apr. 20, 2016.

(51) Int. Cl. G16H 10/60; G16H 10/20; G16H 10/40

(52) U.S. Cl. CPC G16H 10/60; G16H 80/00; G16H 10/40; G16H 10/20

(57) ABSTRACT

Various embodiments described herein relate to methods and apparatuses for documenting data by tracking user interactions with an interface. Users such as medical personnel or the like rely on clinical documentation to treat a patient. By automatically generating clinical documentation based on user interactions with an interface when reviewing patient data, users are not required to spend time in generating clinical documentation themselves.

### TECHNICAL FIELD

Various embodiments described here generally relate to methods and apparatuses for documentation and, more particularly, but not exclusively, to methods and apparatuses for automatic documentation by tracking user interactions with an interface.

### BACKGROUND

Clinical documentation refers to information regarding patient care, such as when a patient is treated in a healthcare institution. Clinical documentation is used, for example, to record a patient's history, record a patient's current complaint, evaluate a patient's current status, develop treatment plans, provide continuity of care, and for other purposes. Typical clinical documentation may contain information related to a patient's vitals, labs, test results, medications, and text information such as history, complaints, diagnosis, and prognosis.

Generating clinical documentation, however, is often a time consuming process. Moreover, increasing patient volumes and higher acuity patients present additional challenges to clinical staff (such as medical personnel) by both increasing the number of notes they have to create and reducing the time to do so.

Clinical documentation becomes even more challenging in intensive care (ICU) where patients are critically ill and require increased attention in a multi-tasking and high-intensity environment. Given the time constraints associated with treatment in ICU, many notes are written after the medical personnel interacts with the patient (either at the end of the personnel's shift or after discharge). Therefore, the documentation relies heavily on the ability of medical personnel to recall important facts, and this can lead to inaccurate and incomplete documentation which negatively affects patient care.

Many healthcare institutions have adopted electronic medical records (EMR) and other types of patient dashboard systems to expedite the note generating process commonly done on paper. However, in these EMR systems, indiscriminate copying and pasting of patient details during the generation of clinical documentation can result in the proliferation of errors. These errors increase the likelihood of patient safety issues when they are acted upon by clinicians.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

Various embodiments relate to a method of documenting data, the method comprising: presenting, via an interface, a plurality of values to a user; storing a first value of the plurality of values for later retrieval in response to the user's interactions with the first value via the interface; and generating at least one report utilizing the stored first value.

These features are advantageous because medical personnel or the like are not required to take the time to generate notes themselves. While reviewing patient data via the interface, the user's interactions with the interface are monitored and information deemed important is then used in a report relating to the patient's condition.

Various embodiments are described wherein the user's interactions with the first value are selected from the group consisting of a mouse event or a keyboard event.

These features are advantageous because a user can easily indicate important information simply by interacting with the data via a keyboard or a mouse.

Various embodiments described herein additionally include storing a second value of the plurality of values for later retrieval in response to the user's interactions with the second value via the interface, wherein the step of generating the at least one report comprises generating at least one report utilizing the stored fist value and the stored second value.

These features are advantageous because the user may interact with two (or more) values that have been presented to “gather” data that is to be used in report generation (e.g., template selection/population).

Various embodiments are described wherein generating the at least one report comprises: retrieving a template from storage; and combining the stored first value with the template.

These features are advantageous because previously stored templates assist in generating clinical notes and ensure compliance with clinical documentation guidelines.

Various embodiments are described wherein combining the stored first value with the template comprises generating at least one natural language statement incorporating the stored first value and situating the at least one generated statement within the template.

These features are advantageous because natural language generation may be used to generate subjective assessments of the patient based on the stored data.

Various embodiments are described wherein storing a first value comprises: retrieving the first data value from a data source; and storing the first data value in a data repository.

These features are advantageous because the first data value, which may be relevant for a patient (as determined by a user interaction with the first data), may be stored to later be used in generating a clinical note.

Various embodiments are described wherein retrieving the first data value from a data source comprises querying a database for the first data value.

These features are advantageous because the stored first value, which may be relevant for a particular patient, may be retrieved from the database when required and used in generating a report.

Various embodiments are described wherein retrieving the first data value from a data source comprises performing character recognition on a screen bitmap to extract the first data value.

These features are advantageous because data can be retrieved more easily and without requiring a user to type or otherwise write the data value in a note themselves.

Various embodiments are described wherein the method further comprises receiving approval from a user of the generated report.

These features are advantageous because it allows the user to ensure the generated report is accurate and/or complete before submitting the report to an archive or other location for further processing, for example.

According to another aspect of the present disclosure, various embodiments relate to an apparatus for documenting data, the apparatus comprising: an interface for presenting a plurality of values to a user; and a processor configured to store a first value of the plurality of values for later retrieval in response to the user's interactions with the first value via the interface; and generate at least one report utilizing the stored first value.

These features are advantageous because medical personnel or the like are not required to take the time to generate notes themselves. While reviewing patient data via the interface, the user's interactions with the interface are monitored and information deemed important is then used in a report relating to the patient's condition.

Various embodiments are described wherein the user's interactions with the first value are selected from the group consisting of a mouse event or a keyboard event.

These features are advantageous because a user can easily indicate important information simply by interacting with the interface via a keyboard or a mouse.

Various embodiments are described wherein the processor is configured to generate the report by retrieving a template from storage and combining the stored first value with the template.

These features are advantageous because previously stored templates assist in generating clinical notes and ensure compliance with clinical documentation guidelines.

Various embodiments are described wherein the processor is configured to combine the stored first value with the template by generating at least one natural language statement incorporating the stored first value and situating the at least one generated statement within the template.

These features are advantageous because natural language generation may be used to generate subjective assessments of the patient based on the stored data.

Various embodiments are described wherein the apparatus further comprises a data source for providing the first data value; and a data repository for storing the first data value.

These features are advantageous because the first data value, which may be relevant for a patient (as determined by a user interaction with the first data value), may be stored to later be used in generating a clinical note.

Various embodiments are described wherein the data source is a database.

This feature is advantageous because data values, indicated as important or otherwise relevant, may be stored in a database for retrieval at a later point in time.

According to yet another aspect of the present disclosure, various embodiments relate to a computer readable medium containing computer-executable instructions for performing a method of documenting data, the medium comprising: computer-executable instructions for presenting, via an interface, a plurality of values to a user; computer-executable instructions storing a first value of the plurality of values for later retrieval in response to the user's interactions with the first value via the interface; and computer-executable instructions for generating at least one report utilizing the stored first value.

These features are advantageous because medical personnel or the like are not required to take the time to generate notes themselves. While reviewing patient data via the interface, the user's interactions with the interface are monitored and information deemed important is then used in a report relating to the patient's condition.

### BRIEF DESCRIPTION OF DRAWINGS

The accompanying drawings are not intended to be drawn to scale. In the drawings, each identical or nearly identical component that is illustrated in various figures may be represented by a like numeral. For purposes of clarity, not every component may be labeled in every drawing. Various embodiments will now be described, by way of example, with reference to the accompanying drawings, in which:

FIG. 1 illustrates an apparatus for documenting data in accordance with one embodiment;

FIG. 2 depicts an exemplary patient record showing patient data in accordance with one embodiment;

FIG. 3 depicts an exemplary log file based on user interactions with the record of FIG. 2 in accordance with one embodiment;

FIG. 4 depicts an example of patient data in accordance with one embodiment;

FIG. 5 depicts an exemplary report based on the data of FIG. 4 in accordance with one embodiment;

FIG. 6 depicts a flowchart of a method of documenting data in accordance with one embodiment;

FIG. 7 depicts a flowchart of a method of documenting data in accordance with another embodiment;

FIG. 8 depicts a flowchart of a method of documenting data in accordance with another embodiment;

FIG. 9 depicts a flowchart of a method of documenting data in accordance with yet another embodiment; and

FIG. 10 presents a system for documenting data in accordance with one embodiment.

### DETAILED DESCRIPTION

To address the problems associated with EMR, many clinical documentation solutions have been introduced into the healthcare environment. These solutions range from the automated conversion of paper clinical notes to an electronic format (by scanning) to the automated dictation of patient summaries. However, there remains room for improvement in terms of eliminating the time constraints associated with clinical documentation generation and the likelihood of propagating significant errors in clinical narratives still persist.

It would be desirable, therefore, for documentation methods and apparatuses that more fully address these concerns.

Various embodiments are described more fully below with reference to the accompanying drawings, which form a part hereof, and which show specific exemplary embodiments. However, the concepts of the present disclosure may be implemented in many different forms and should not be construed as limited to the embodiments set forth herein; rather, these embodiments are provided as part of a thorough and complete disclosure, to fully convey the scope of the concepts, techniques and implementations of the present disclosure to those skilled in the art. Embodiments may be practiced as methods, systems or devices. Accordingly, embodiments may take the form of a hardware implementation, an entirely software implementation (running on hardware such as a processor) or an implementation combining software and hardware aspects. The following detailed description is, therefore, not to be taken in a limiting sense.

Reference in the specification to “one embodiment” or to “an embodiment” means that a particular feature, structure, or characteristic described in connection with the embodiments is included in at least one example implementation or technique in accordance with the present disclosure. The appearances of the phrase “in one embodiment” in various places in the specification are not necessarily all referring to the same embodiment.

Some portions of the description that follow are presented in terms of symbolic representations of operations on non-transient data stored within a computer memory. These descriptions and representations are used by those skilled in the data processing arts to most effectively convey the substance of their work to others skilled in the art. Such operations typically require physical manipulations of physical quantities. Usually, though not necessarily, these quantities take the form of electrical, magnetic or optical signals capable of being stored, transferred, combined, compared and otherwise manipulated. It is convenient at times, principally for reasons of common usage, to refer to this data as bits, values, elements, symbols, characters, terms, numbers, or the like. Furthermore, it is also convenient at times, to refer to certain arrangements of steps requiring physical manipulations of physical quantities as modules or code devices, without loss of generality.

However, all of these and similar terms are to be associated with the appropriate physical quantities and are merely convenient labels applied to these quantities. Unless specifically stated otherwise as apparent from the following discussion, it is appreciated that throughout the description, discussions utilizing terms such as “processing” or “computing” or “calculating” or “determining” or “displaying” or the like, refer to the action and processes of a computer system, or similar electronic computing device, that manipulates and transforms data represented as physical (electronic) quantities within the computer system memories or registers or other such information storage, transmission or display devices. Portions of the present disclosure include processes and instructions that may be embodied in software, firmware or hardware, and when embodied in software, may be downloaded to reside on and be operated from different platforms used by a variety of operating systems.

The present disclosure also relates to an apparatus for performing the operations herein. This apparatus may be specially constructed for the required purposes, or it may comprise a general-purpose computer selectively activated or reconfigured by a computer program stored in the computer. Such a computer program may be stored in a non-transitory computer readable storage medium, such as, but is not limited to, any type of disk including floppy disks, optical disks, CD-ROMs, magnetic-optical disks, read-only memories (ROMs), random access memories (RAMs), EPROMs, EEPROMs, magnetic or optical cards, application specific integrated circuits (ASICs), or any type of media suitable for storing electronic instructions, and each may be coupled to a computer system bus. As used herein, the term “non-transitory computer-readable storage medium” will be understood to encompass both volatile and non-volatile memories, but to exclude transitory signals. Furthermore, the computers referred to in the specification may include a single processor or may be architectures employing multiple processor designs for increased computing capability.

The processes and displays presented herein are not inherently related to any particular computer or other apparatus. Various general-purpose systems may also be used with programs in accordance with the teachings herein, or it may prove convenient to construct more specialized apparatus to perform one or more method steps. The structure for a variety of these systems is discussed in the description below. In addition, any particular programming language that is sufficient for achieving the techniques and implementations of the present disclosure may be used. A variety of programming languages may be used to implement the present disclosure as discussed herein.

In addition, the language used in the specification has been principally selected for readability and instructional purposes and may not have been selected to delineate or circumscribe the disclosed subject matter. Accordingly, the present disclosure is intended to be illustrative, and not limiting, of the scope of the concepts discussed herein.

Various features of embodiments described herein leverage patient dashboards and EMR interfaces that may display patient information grouped into categories. The apparatus can track the user's behavior when viewing this information (e.g., by user cursor movement and clicks) as they browse the data displayed on the patient dashboard. Based on the information selected by the user, the apparatus may generate an at least partially complete note with values such as vital signs, lab values, and medications, for example.

In the context of the application, the term “user” may refer to any type of clinician or medical personnel such as a doctor, physician, nurse, therapist, administrative assistant, or other interested party providing patient care.

Although some features of the embodiments described herein are described as being implemented in healthcare applications, it is contemplated that they may be implemented in other applications. For example, the features may be used in financial market analysis, manufacturing operations, logistic operations, climate analysis, counter-terrorism/crime prevention, or any other application that relies on the analysis of data to generate reports.

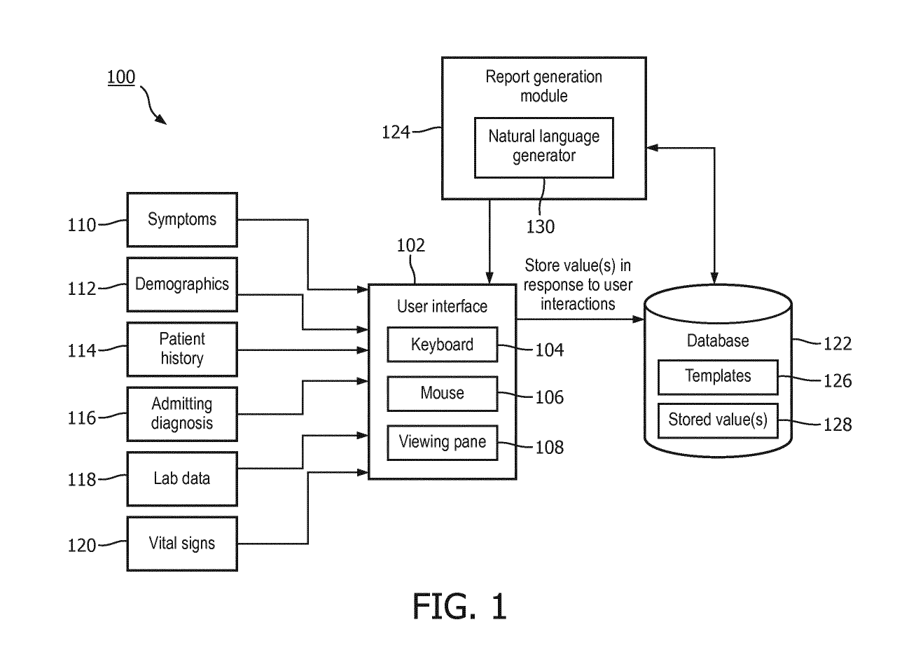

FIG. 1 depicts an apparatus 100 for documenting data in accordance with one embodiment. The apparatus 100 may include a user interface 102 with a keyboard 104, a mouse 106 (or other type of gestural input, e.g., stylus), and a viewing pane 108 to enable a user to view and interact with the presented patient data via an EMR or other type of patient dashboard. Various alternative devices for implementing the user interface 102 will be apparent; for example, the user interface 102 may be implemented by a smart phone or tablet in some embodiments. The patient data may be communicated to the user interface 102 from a variety of data sources, and may include information related to symptoms 110, demographics 112, patient history 114, admitting diagnosis 116, lab data 118, and vital signs 120.

The apparatus 100 may further include a database 122 and a report generation module 124. The database 122 may store templates 126 used in report generation to, for example, ensure compliance with clinical documentation guidelines. The database 122 may also store data values 128 based on the user's interactions via the user interface 102, copies of various values obtained from one or more data sources, intermediary results produced when merging patient data with templates, draft reports, etc.

The report generation module 124 may be used to generate clinical documentation (e.g., a report on a patient) based on the stored data. The report generation module 124 may include a natural language generator 130 to provide natural language assessments of a patient in the generated report.

The user interface 102 may be implemented as a PC, laptop, smartphone, tablet, or the like. The user interface 102 may display patient data and other information in the form of tables, graphs, color schemes, or in any other format convenient for a user. The user interface 102 may receive information relating to the patient via any hardwired or wireless connection.

The symptoms 110 may be described by the patient to medical personnel at the time of patient check-in. The symptoms 110 may also include any characteristics of the patient observed by medical personnel.

Demographics 112 may include information previously known by the healthcare institution or medical personnel, or information obtained at the time of patient admission. The demographics may include, for example, patient age, gender, height, weight, body mass index, etc.

Patient history 114 may include information previously known by the healthcare institution, or information obtained at the time of patient admission. Patient history 114 may include information such as previous patient diagnoses, patient family history, allergies, whether the patient has traveled abroad recently, etc.

Admitting diagnosis 116 may be the initial diagnosis at the time of admission and may be based on at least the symptoms 110, demographics 112, and patient history 114. The diagnosis 116 may be made by medical personnel such as a doctor, nurse, physician, or the like.

Lab data 118 may include the results of certain tests performed on the patient. The laboratory data 118 obtained may depend on the test(s) performed, which may in turn vary depending on the patient and their health status.

Vital signs 120 may be obtained autonomously or by medical personnel. The vital signs 120 may be gathered via appropriate sensor devices and may include arterial (systolic and diastolic) blood pressure, heart rate, respiratory rate, temperature, O₂ saturation (e.g., arterial oxygen saturation (SpO₂)), or the like. The vital signs 120 gathered may vary and may depend on the particular patient and their health status.

The database 122 may include one or more non-transitory machine-readable storage media such as read-only memory (ROM), random-access memory (RAM), magnetic disk storage media, optical storage media, flash-memory devices, or similar storage media. In various embodiments, the database 122 may store instructions for execution by the report generation module 124 or data upon with the report generation module 124 may operate.

The report generation module 124 may be any specifically configured processor or hardware device capable of generating the report using the previously stored templates and stored data values. The report generation module 124 may include a microprocessor, a field programmable gate array (FPGA), application-specific integrated circuit (ASIC), or other similar device(s). In some embodiments, such as those relying on one or more ASICs, the functionality described as being provided at least in part via software may instead be configured into the design of the ASICs, and as such, any associated software may be omitted.

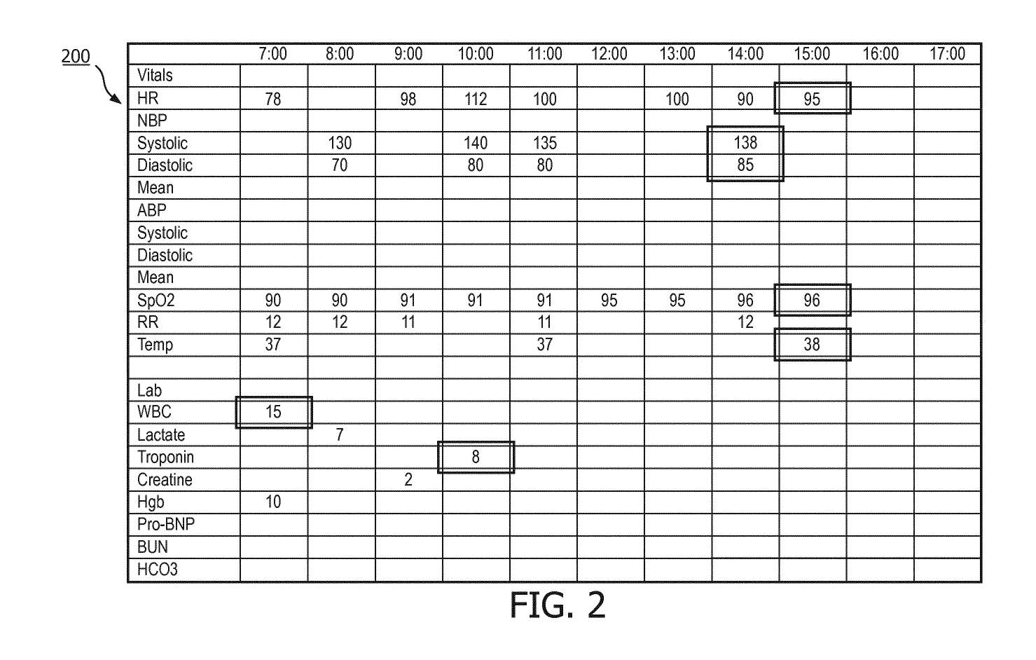

In operation, a user such as medical personnel or the like may view patient data via the user interface 102. For example, patient data may be presented in the form of an EMR such as the table 200 of FIG. 2. A user, via their keyboard 104, mouse 106, and/or viewing pane 108 may view and interact with the table 200 to select certain data. The interaction with data can occur in the course of performing diagnostics, administering treatment, etc., or it may explicitly be used in generating a report.

For example, a user may hover their cursor, via the mouse 106, over a data field or may hold a stylus on a data field for a predetermined period of time (e.g., for at least two seconds) to select a piece of data that is potentially important. Or, a user may use the directional arrow keys on their keyboard to highlight a data field for a predetermined period of time. As yet another example, a user may scroll or otherwise view, via the viewing pane 108, certain pieces of data for a predetermined period of time. As still another example, a user may select a data item using a mouse 106 and actuating one of the buttons on the mouse 106.

FIG. 2, for example, shows that certain data fields are highlighted as a result of interaction events between the user and the user interface 102. These highlights may actually be displayed on the interface 102, or they may be invisible to the user, in the sense that they denote for purposes of this discussion that the user has interacted with that specific data field. This figure uses a generic single patient viewer as an example of the patient dashboard. The user may have interacted with the data shown in the highlighted boxes either by, e.g., clicking on them or hovering over them for a certain period of time.

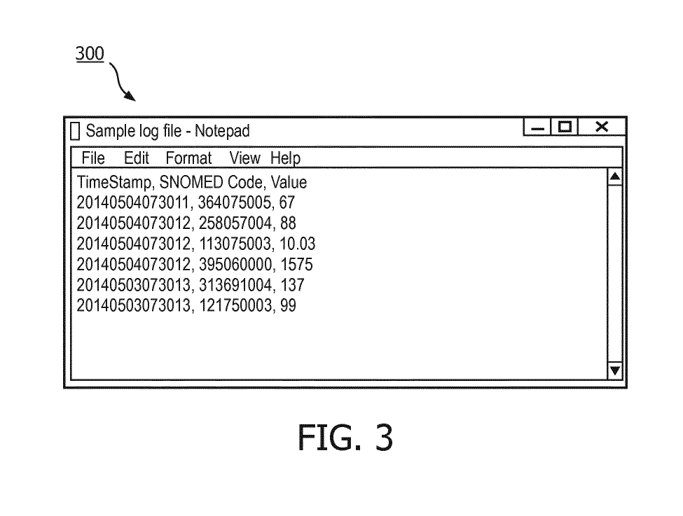

As the user browses through the patient dashboard or other patient record, the viewed (e.g., selected) data may be saved in the form of a log file such as the log file 300 of FIG. 3. The log file 300 may save all data values the user interacted with (e.g., data that the user selected) and may contain information on the date and time the data was selected, along with the type of data and its value.

As mentioned previously, the database 122 also stores predefined templates 126 that are used in the report generation process. There may be several different templates stored in the database 122, each for a different purpose. The template used to generate a report may be selected by a user or, depending on the type (e.g., category) of data viewed/selected by user, a certain template may automatically be selected.

For example, the database 122 may store templates for daily progress notes and templates for consultation notes. Each template may be programmed to contain some required types of data (e.g., based on documentation guidelines), which may be included regardless of whether certain information was selected or otherwise viewed by the user.

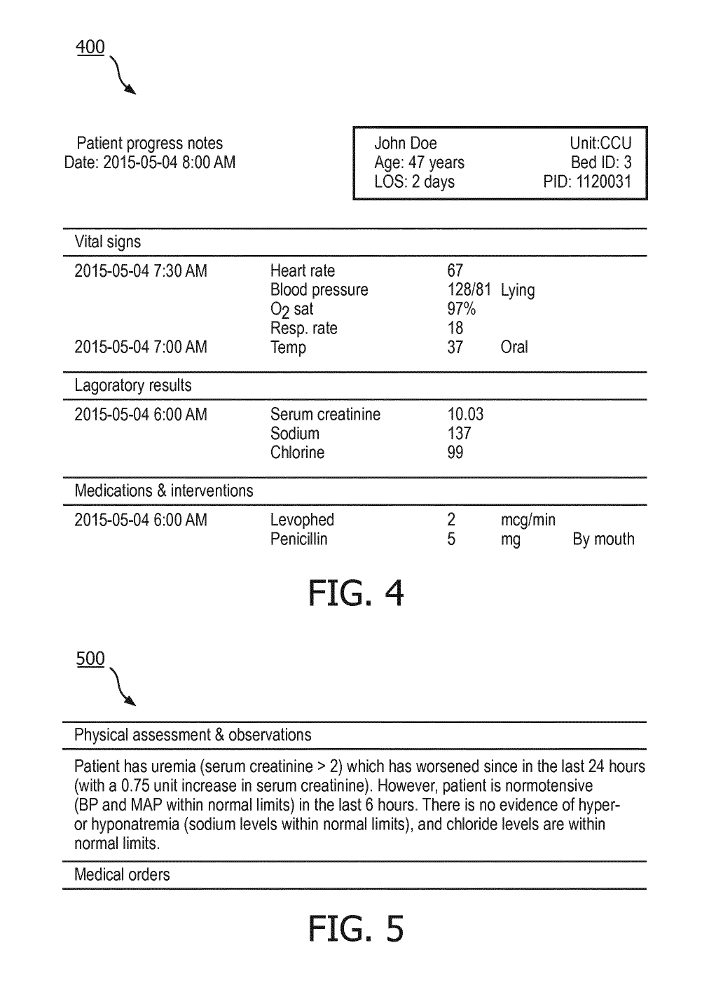

Referring back to FIG. 1, the report generation module 124 may query the database 122 for a certain template and populate the selected template with the stored data value(s). FIG. 4, for example, presents a template 400 populated with vital signs; laboratory results; medications; and interventions derived from the data in the log file of FIG. 3. This particular template 400 may have automatically been selected from the database 122 based on the information selected by the user (e.g., if the user interacted with vital sign and/or laboratory results data). By considering only information relevant to the user, the database 122 inevitably uses less memory for storage and the report generation module 124 requires less processing power.

It is noted that some sections in a typical clinical note are specified and quantifiable (such as vital signs, lab data, and medications), while other sections consist of free text which may include observations, trends, etc. The free text portion may be created by using the natural language generator 130. The natural language generator 130 may, for example, interpret data values viewed by the user, and construct complete sentences discussing trends, conclusions, recommendations for treatment, etc.

FIG. 5, for example, depicts an exemplary report 500 in accordance with one embodiment. As shown, the report generation module 124 has analyzed the stored data values and, via the natural language generator 130, has developed an assessment regarding the patient. For example, the assessment discusses that the patient has uremia (based on serum creatinine levels) that has recently worsened.

In other embodiments, for example, the natural language generator 130 may generate a sentence such as “patient's laboratory results reveals hyperkalemia and azotemia” if it detects that the log data from the user tracking contains a value for potassium (K+) greater than 5 mEq/L and a value for BUN (Blood Urea Nitrogen) greater than 20 mg/dl (or creatinine levels greater than 1.3 mg/dl). Natural language generation may also be used in assessments of patient trends, e.g., if the patient's heart rate has been increasing in the last 6 hours.

The generated note may then be presented to the user via the user interface 102, who may then fill in other sections and edit the pre-filled sections. Once the user has finished editing the report and saved it, the note will be archived appropriately. The user may also opt to turn off user tracking. At this point, the user interface 102 may give the user the option to view other data and/or to create a new note.

The saved notes, as well as the user's edits to the notes, can also be used in generating future notes. For example, the report generation module 124 may note the changes made by the user to the generated note during editing. The report generation module 124 may then use these learned patterns to create a more tailored assessment for future notes for a particular user, to revise an existing template, or to create a new template.

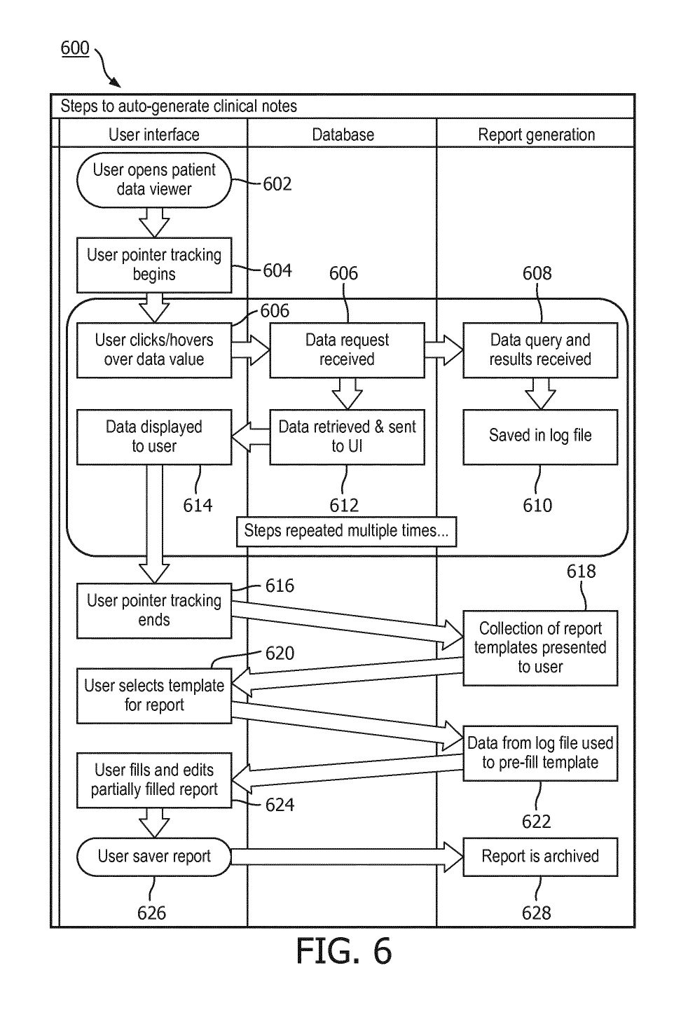

FIG. 6 depicts a flowchart of a method 600 of documenting data in accordance with one embodiment. A user may open a patient dashboard 602 (e.g., an EMR) via the user interface 102, at which point the user's interactions with the dashboard will be tracked 604. It is contemplated that the user can be tracked from the moment they log into the patient dashboard, or the user may have the option to select when he or she would like to be tracked. This feature gives the user more control over the process as the user can browse only relevant information for the note during the tracking period.

Information the user selects 606 (e.g., information the user interacts with through user clicks or by hovering over the information), is recorded and communicated 606 to the database 122. The information remains stored until it is requested 608 by the report generation module 124. The report generation module may query the database 608 once the user indicates they are done viewing data or otherwise requests the generation of a report. Once queried and received 608, the data may be saved in a log file 610 such as the log file 300 of FIG. 3. It is also noted that the user interface 102 may display data 614 that the user indicated as important (which may retrieved and sent 612 to the UI by the database) to assure the user the data has been selected (e.g., by highlighting the data as shown in FIG. 2).

Once the user indicates they are done viewing data 616, the tracking will end and the user is presented with a collection of report templates 618. The user may then select a template 620 to be used to generate the final report. The user's selection may be made based on personal preference and may depend on the particular types of data to be used in the generated report.

Once the user selects a template 620, the stored data is used to pre-fill the template 622. Additionally, the natural language generator 130 may add textual information, such as assessments and recommendations. The user may then have to option to edit the partially filled (or complete) report 624. The user may then save the report 626, at which point the report is archived 628.

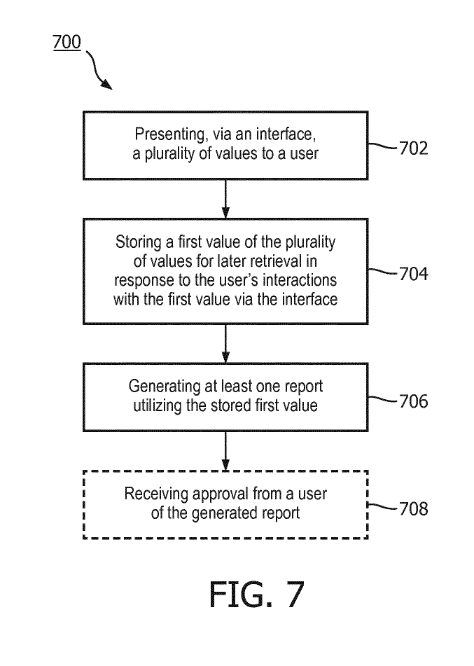

FIG. 7 depicts a flowchart of a method 700 of documenting patient data in accordance with another embodiment. Step 702 involves presenting, via an interface, a plurality of values to a user. The interface may be the user interface of FIG. 1, for example, and the plurality of values may relate to patient health and may be presented in the form of a table such as the table 200 of FIG. 2.

Step 704 involves storing a first value of the plurality of values for later retrieval in response to the user's interactions with the first value via the interface. The first value may be stored in the database 122, for example.

Step 706 involves generating at least one report utilizing the stored first value. The report may be generated by the report generation module 124, for example.

Step 708 is optional, and involves receiving approval from a user of the generated report. After the report is generated, the user may have the opportunity to review the generated report for completeness and/or accuracy. The user may further edit the report by entering additional information and/or deleting certain information. When the user is satisfied with the generated report, the user may approve the report and the report may be archived for later use or it may be discarded.

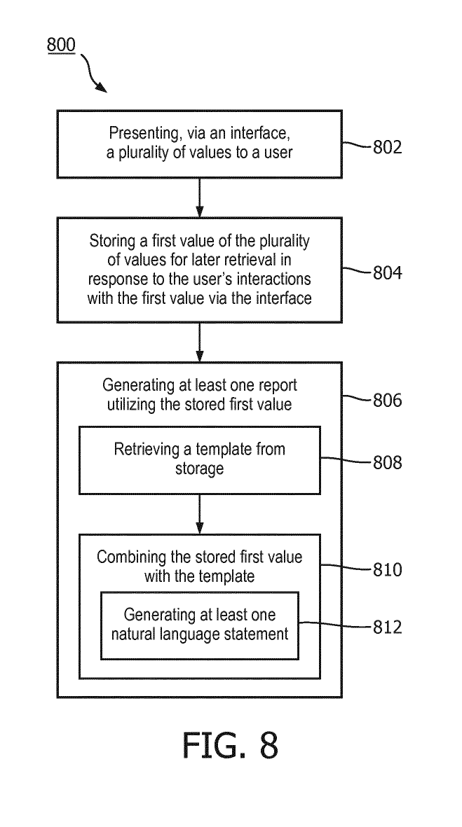

FIG. 8 depicts a flowchart of a method 800 of documenting patient data in accordance with another embodiment. Steps 802 and 804 are similar to steps 702 and 704, respectively, of FIG. 7 and are not repeated here.

Step 806 involves generating at least one report utilizing the stored first value, and comprises steps 808 and 810. Step 808 involves retrieving a template from storage. The storage may be the database 122, for example. The particular template retrieved from storage may depend on the particular type (e.g., category) of the stored data value(s).

Step 810 involves combining the stored first value with the template. This step may be performed by the report generation module 124. In some embodiments, step 810 may include the additional step 812 of generating at least one natural language statement. This step may be performed by the natural language generator 130, for example. The at least one generated statement may include information such as whether a patient's condition is improving or worsening, reasons why a patient may be developing a certain condition, and recommendation(s) for treatment, for example.

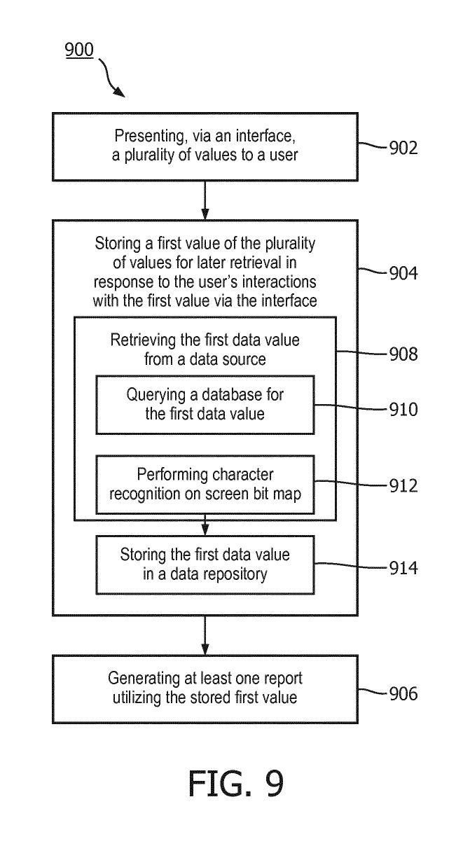

FIG. 9 depicts a flowchart of a method 900 of documenting data in accordance with another embodiment. Steps 902 and 906 are similar to steps 702 and 706, respectively, of FIG. 7 and are not repeated here.

Step 904 involves storing a first value of the plurality of values for later retrieval in response to the user's interactions with the first value via the interface, and includes steps 908 (with steps 910 and 912) and 914.

Step 908 involves retrieving the first data value from a data source and consists of step 910 that involves querying a database for the first data value. This may be a database storing information related to a specific patient (e.g., patient lab results). Step 912 involves performing character recognition on a screen bit map. In this embodiment, the apparatus 100 may further include, for example, an optical character recognition engine for recognizing characters (data values) in the patient dashboard.

Step 914 involves storing the first data value in a data repository. This data repository may be the database 122, for example.

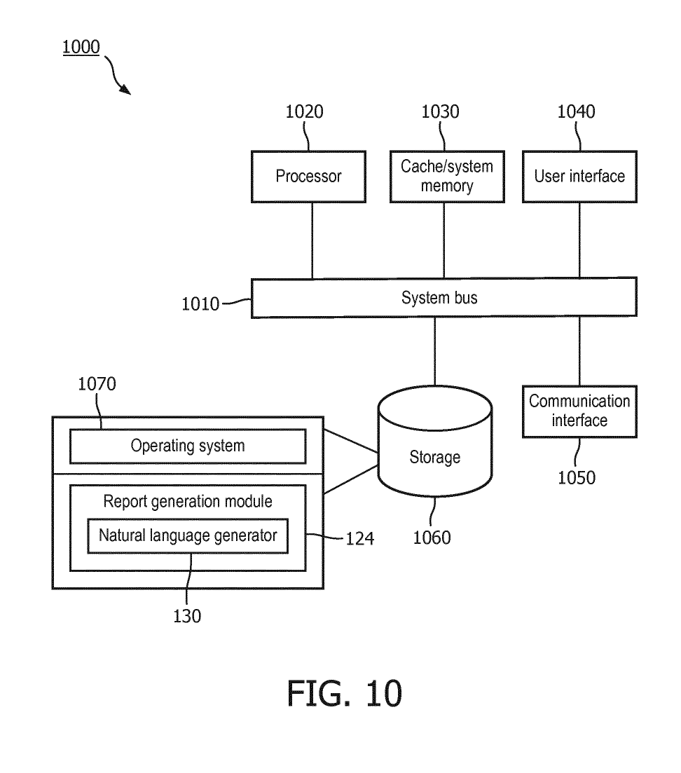

FIG. 10 illustrates an example of a hardware system 1000 for implementing various devices that may participate in the various methods described herein. For example, the hardware system 1000 may implement the report generation module 124 of FIG. 1 and may communicate with a physically separate user interface 102 and/or database 122. In other embodiments, the user interface 102 and/or database 122 may be implemented together with the report generation module 124 on the hardware system 124. In some embodiments, the report generation module 124 (and/or other devices described herein) may be implemented within a cloud computing architecture; as such, the hardware system 1000 may be or may be virtually implemented by hardware resident within or among one or more cloud computing data centers while the report generation module 124 (and/or other device) may be implemented as part of a virtual machine running on such hardware.

As shown in FIG. 10, the hardware 1000 includes one or more system buses 1010 that connect a processor 1020, cache/system memory 1030, a user interface 1040, a communication interface 1050, and storage 1060. It will be understood that FIG. 10 is merely exemplary and constitutes, in some respects, an abstraction and that the actual organization of the components of the hardware 1000 may vary and be more complex than illustrated.

The processor 1020 may be any hardware device capable of executing instructions stored in memory 1030 or storage 1060 or otherwise processing data. As such, the processor 1020 may include a microprocessor, a field programmable gate array (FPGA), application-specific integrated circuit (ASIC), or other similar devices. In some embodiments, such as those relying on one or more ASICs, the functionality described as being provided in part via software may instead be configured into the design of the ASICs and, as such, the associated software may be omitted.

The cache/system memory 1030 may include various memories such as, for example, L1, L2, or L3 cache or system memory. As such, the memory 1030 may include static random access memory (SRAM), dynamic RAM (DRAM), flash memory, read only memory (ROM), or other similar memory devices.

The user interface 1040 may include one or more devices for enabling communication with a user such as medical personnel. For example, the user interface 1040 may include a display, a mouse, a keyboard, a touchscreen, buttons, camera, microphone, vibrator, haptic engine, etc. In some embodiments, the user interface 1040 may include a command line interface or graphical user interface that may be presented to a remote terminal via the communication interface 1050.

The communication interface 1050 may include one or more devices for enabling communication with other hardware devices. For example, the communication interface 1050 may include a network interface card (NIC) configured to communicate according to WiFi or Ethernet protocols. Additionally the communication interface 1050 may implement a TCP/IP stack for communicating according to the TCP/IP protocols. In some embodiments, the communication interface 1050 may include an NFC, Bluetooth, or other short range wireless interface. Various alternative or additional hardware or configurations for the communication interface 1050 will be apparent.

The storage 1060 may include one or more machine-readable storage media such as read-only memory (ROM), random-access memory (RAM), magnetic disk storage media, optical storage media, flash-memory devise, or similar storage media. In various embodiments, the storage 1060 may store instructions for execution by the processor 1020 or data upon which the processor 1020 may operate. For example, the storage 1060 may store an operating system 1070 for controlling various basic operations of the hardware system 1000. These operations may include generating reports by the report generation module 124 and generating natural language statements by the natural language generator 130 such as those illustrated in FIG. 1.

The methods, systems, and devices discussed above are examples. Various configurations may omit, substitute, or add various procedures or components as appropriate. For instance, in alternative configurations, the methods may be performed in an order different from that described, and that various steps may be added, omitted, or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Embodiments of the present disclosure, for example, are described above with reference to block diagrams and/or operational illustrations of methods, systems, and computer program products according to embodiments of the present disclosure. The functions/acts noted in the blocks may occur out of the order as shown in any flowchart. For example, two blocks shown in succession may in fact be executed substantially concurrent or the blocks may sometimes be executed in the reverse order, depending upon the functionality/acts involved. Additionally, or alternatively, not all of the blocks shown in any flowchart need to be performed and/or executed. For example, if a given flowchart has five blocks containing functions/acts, it may be the case that only three of the five blocks are performed and/or executed. In this example, any of the three of the five blocks may be performed and/or executed.

A statement that a value exceeds (or is more than) a first threshold value is equivalent to a statement that the value meets or exceeds a second threshold value that is slightly greater than the first threshold value, e.g., the second threshold value being one value higher than the first threshold value in the resolution of a relevant system. A statement that a value is less than (or is within) a first threshold value is equivalent to a statement that the value is less than or equal to a second threshold value that is slightly lower than the first threshold value, e.g., the second threshold value being one value lower than the first threshold value in the resolution of the relevant system.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Having described several example configurations, various modifications, alternative constructions, and equivalents may be used without departing from the spirit of the disclosure. For example, the above elements may be components of a larger system, wherein other rules may take precedence over or otherwise modify the application of various implementations or techniques of the present disclosure. Also, a number of steps may be undertaken before, during, or after the above elements are considered.

Having been provided with the description and illustration of the present application, one skilled in the art may envision variations, modifications, and alternate embodiments falling within the general inventive concept discussed in this application that do not depart from the scope of the following claims.

1. A method of documenting data, the method comprising:
presenting, via an interface, a plurality of values to a user;
storing a first value of the plurality of values for later retrieval in response to the user's interactions with the first value via the interface; and
generating at least one report utilizing the stored first value,
wherein generating the at least one report comprises:
retrieving a template from storage based on the stored first value, and
combining the stored first value with the template.

2. The method of claim 1, wherein the user's interactions with first value are selected from the group consisting of a mouse event or a keyboard event.

3. The method of claim 1, further comprising:
storing a second value of the plurality of values for later retrieval in response to the user's interactions with the second value via the interface,
wherein the step of generating the at least one report comprises generating at least one report utilizing the stored fist value and the stored second value.

4. (canceled)

5. The method of claim 1, wherein combining the stored first value with the template comprises generating at least one natural language statement incorporating the stored first value and situating the at least one generated statement within the template.

6. The method of claim 1, wherein storing a first value comprises:
retrieving the first data value from a data source; and
storing the first data value in a data repository.

7. The method of claim 6, wherein retrieving the first data value from a data source comprises querying a database for the first data value.

8. The method of claim 6, wherein retrieving the first data value from a data source comprises performing character recognition on a screen bitmap to extract the first data value.

9. The method of claim 1, further comprising receiving approval from a user of the generated report.

10. An apparatus for documenting data, the apparatus comprising:
an interface for presenting a plurality of values to a user; and
a processor configured to:
store a first value of the plurality of values for later retrieval in response to the user's interaction with the first value via the interface; and
generate at least one report utilizing the stored first value by retrieving a template from storage based on the stored first value and combining the stored first value with the template.

11. The apparatus of claim 10, wherein the user's interactions with the first value are selected from the group consisting of a mouse event or a keyboard event.

12. The apparatus of claim 10, wherein the processor is further configured to:
storing a second value of the plurality of values for later retrieval in response to the user's interactions with the second value via the interface,
wherein the step of generating the at least one report comprises generating at least one report utilizing the stored fist value and the stored second value.

13. (canceled)

14. The apparatus of claim 10, wherein the processor is configured to combine the stored first value with the template by generating at least one natural language statement incorporating the stored first value and situating the at least one generated statement within the template.

15. The apparatus of claim 10, further comprising a data source for providing the first data value; and a data repository for storing the first data value.

16. The apparatus of claim 15, wherein data source is a database.

17. A non-transitory computer readable medium containing computer-executable instructions for performing a method of documenting data, the medium comprising:
computer-executable instructions for presenting, via an interface, a plurality of values to a user;
computer-executable instructions storing a first value of the plurality of values for later retrieval in response to the user's interactions with the first value via the interface; and
computer-executable instructions for generating at least one report utilizing the stored first value, wherein generating the at least one report comprises:
receiving a template from storage based on the stored first value; and
combining the stored first value with the template.
